(19) United States
(12) Patent Application Publication
(10) Pub. No.: US 2021/0290445 A1
(43) Pub. Date: Sep. 23, 2021

(54) COMPRESSION PRODUCT FOR THE TREATMENT OF OSAHS

(71) Applicant: LABORATOIRES INNOTHERA, Arcueil (FR)

(72) Inventors: Nicolas Patrikeeff, Saint Vallier (FR); Dominico Demontis, Frouard (FR); François Cros, Ivry Sur Seine (FR); Amina Ouchene, Creteil (FR)

(21) Appl. No.: 17/202,695

(22) Filed: Mar. 16, 2021

(30) Foreign Application Priority Data
Mar. 18, 2020 (FR) 2002651

(51) Int. Cl. A61F 13/08

(52) U.S. Cl. CPC A61F 13/08

(57) ABSTRACT

An elastic compression product for a wearer's lower limbs, intended for the treatment of obstructive sleep apnoea-hypopnea syndrome, the product having a pressure profile defining textile pressures P_{B}, P_{B1}, P_{C}, P_{D}, P_{E}, P_{F}, P_{G}, P_{H}, P_{I}, P_{D′}, P_{E′}, P_{F′} and P_{G′} at altitudes B, B1, C, D, E, F, G, H, I defined in section 4.1.2 of standard NF_S_97_114, and at altitudes D′, E′, F′ and G′ located respectively at 92% of altitude D, at 110% of altitude E, at 126% of altitude E and at 92% of altitude G, respectively, the pressures being measured as the compression pressures of compression stockings according to French standard NF G 30-102 part B, the pressure profile being such that
30 mmHg>P_{C}>10 mmHg,
30 mmHg>P_{F}>10 mmHg and
P_{C}>(P_{B}+10 mmHg).

### TECHNICAL FIELD

The present invention relates to compression products for the treatment of obstructive sleep apnoea/hypopnea syndrome (OSAHS).

### BACKGROUND

OSAHS is characterized by the occurrence, during sleep, of abnormally frequent episodes of interruption of ventilation (apnoeas), or of significant reduction in ventilation (hypopneas). It is linked to a repeated collapse of the upper airway during sleep. Episodes of apnoea and of hypopnea lead to hypoxemia and micro-awakenings.

The severity of OSAHS has been defined by the Société de pneumologie de langue frangaise (SPLF) based on the following two components:
the severity of daytime sleepiness (mild, moderate, severe), and
the apnoea-hypopnea index (AHI), defined by the number of apnoeas or hypopneas recorded per hour of sleep (mild: 5<AHI<15; moderate: 15<AHI<30; severe: AHI>30).

In 2011, Redolfi et al. report in the publication titled “Attenuation of obstructive sleep apnea by compression stockings in subjects with venous insufficiency” that, in 12 non-obese subjects with venous insufficiency and OSAHS having an AHI >15, the daytime wearing for one week of thigh-length compression stockings applying 20 mmHg to the ankle reduced the AHI by 36% on average, the nocturnal decrease in leg fluid volume by 62% and the nocturnal increase in cervical perimeter by 60%.

In 2015, White et al. published a paper titled “Effect of below-the-knee compression stockings on severity of obstructive sleep apnea” which showed similar results.

There is an ongoing need to improve compression products for the treatment of OSAHS.

One aim of the invention is to meet, at least partially, this need.

### DISCLOSURE OF THE INVENTION

The invention proposes an elastic compression product for a wearer's lower limbs, intended for the treatment of obstructive sleep apnoea/hypopnea syndrome, said product having a pressure profile defining textile pressures P_{B}, P_{B1}, P_{C}, P_{D}, P_{E}, P_{F}, P_{G}, P_{H}, P_{I}, P_{D′}, P_{E′}, P_{F′} and P_{G′} at altitudes B, B1, C, D, E, F, G, H, I defined in section 4.1.2 of standard NF_S_97_114, and at altitudes D′, E′, F′ and G′ located respectively at 92% of altitude D, at 110% of altitude E, at 126% of altitude E and at 92% of altitude G, respectively, the pressures being measured as the compression pressures of compression stockings according to French standard NF G 30-102 part B, the pressure profile being such that
35 mmHg>P_{C}>10 mmHg,
30 mmHg>P_{F}>10 mmHg and
P_{C}>(P_{B}+10 mmHg).

Surprisingly, the pressure profile of a compression product according to the invention reduces the AHI.

A product according to the invention may also have one or more of the following optional and preferred features:
the pressure profile has
   a local minimum pressure at altitude B, and/or
   a local maximum pressure between altitudes B1 and D′, and/or
   a local maximum pressure between altitudes F′ and G′, and/or
   a local minimum pressure between altitudes D and E′, and/or
   a local minimum pressure between altitudes H and I;
P_{C}>(P_{F}+8 mmHg);
33 mmHg>P_{C}>25 mmHg, preferably 32 mmHg>P_{C}>26 mmHg, or even 30 mmHg>P_{C}>28 mmHg;
3 mmHg<P_{B}<15 mmHg, and
P_{E}<13 mmHg, and
P_{H}<10 mmHg;
5 mmHg<P_{B}<13 mmHg, and
P_{E}<13 mmHg, and
P_{H}<10 mmHg;
P_{C}>20 mmHg; and
mmHg>P_{F}>10 mmHg and
P_{C}>(P_{B}+10 mmHg);
10 mmHg>P_{B}>2 mmHg, and/or
15 mmHg>P_{D}>5 mmHg, and/or
13 mmHg>P_{E}>5 mmHg, and/or
15 mmHg>P_{E′}>5 mmHg, and/or
10 mmHg>P_{I}>2 mmHg;
30 mmHg>P_{B1}>15 mmHg, preferably 28 mmHg>P_{B1}>18 mmHg, and/or
30 mmHg>P_{D′}>15 mmHg, preferably 28 mmHg>P_{D′}>18 mmHg, and/or
25 mmHg>P_{F′}>10 mmHg, preferably 23 mmHg>P_{F′}>13 mmHg, or even
20 mmHg>P_{F′}, or even 18 mmHg>P_{F′}, and/or
25 mmHg>P_{G′}>10 mmHg, preferably 23 mmHg>P_{G′}>13 mmHg, or even
20 mmHg>P_{G′}, or even 18 mmHg>P_{G′};
the product does not extend beyond altitude I;
the product is in the form of a kit including a pair of socks and a pair of shorts;
the kit includes one or more of the following optional and preferred features:
   15 mmHg>P_{B}>3 mmHg, and/or
   35 mmHg>P_{B1}>20 mmHg, and/or
   35 mmHg>P_{C}>20 mmHg, and/or
   15 mmHg>P_{D}>5 mmHg, and/or
   23 mmHg>P_{F}>15 mmHg, and/or
   20 mmHg>P_{G}>10 mmHg, and/or
   10 mmHg>P_{H}>2 mmHg, and/or
   10 mmHg>P_{I}>2 mmHg, and preferably
   35 mmHg>P_{D′}>25 mmHg, and/or
   15 mmHg>P_{E′}>5 mmHg, and/or
   23 mmHg>P_{F′}>15 mmHg, and/or
   23 mmHg>P_{G′}>15 mmHg;
the kit also includes one or more of the following optional and preferred features:
   15 mmHg>P_{B}>3 mmHg, and
   35 mmHg>P_{B1}>20 mmHg, and
   35 mmHg>P_{C}>20 mmHg, and
   15 mmHg>P_{D}>5 mmHg, and
   23 mmHg>P_{F}>15 mmHg, and
   20 mmHg>P_{G}>10 mmHg, and
   10 mmHg>P_{H}>2 mmHg, and
   10 mmHg>P_{I}>2 mmHg. and preferably
   35 mmHg>P_{D′}>25 mmHg, and
   15 mmHg>P_{E′}>5 mmHg, and
   23 mmHg>P_{F′}>15 mmHg, and
   23 mmHg>P_{G′}>15 mmHg;
the kit includes one or more of the following optional and preferred features:
   12 mmHg>P_{B}>5 mmHg, and/or
   30 mmHg>P_{B1}>25 mmHg, and/or
   30 mmHg>P_{C}>25 mmHg, and/or
   12 mmHg>P_{D}>8 mmHg, and/or
   20 mmHg>P_{E}>15 mmHg, and/or
   17 mmHg>P_{G}>13 mmHg, and/or
   7 mmHg>P_{H}>3 mmHg, and/or
   7 mmHg>P_{I}>3 mmHg, and preferably
   30 mmHg>P_{D′}>26 mmHg, and/or
   12 mmHg>P_{E′}>8 mmHg, and/or
   20 mmHg>P_{F′}>15 mmHg, and/or
   20 mmHg>P_{G′}>15 mmHg:
the kit also includes one or more of the following optional and preferred features:
   12 mmHg>P_{B}>5 mmHg, and
   30 mmHg>P_{B1}>25 mmHg, and
   30 mmHg>P_{C}>25 mmHg, and
   12 mmHg>P_{D}>8 mmHg, and
   20 mmHg>P_{E}>15 mmHg, and
   17 mmHg>P_{F}>13 mmHg, and
   7 mmHg>P_{G}>3 mmHg, and
   7 mmHg>P_{I}>3 mmHg, and preferably
   30 mmHg>P_{D′}>26 mmHg, and
   12 mmHg>P_{E′}>8 mmHg, and
   20 mmHg>P_{F′}>15 mmHg. and
   20 mmHg>P_{G′}>15 mmHg;
the product has the shape of a legging, i.e., a one-piece compression product extending at least from the ankles to the hips;
the legging, according to a first embodiment, has one or more of the following optional and preferred features:
   10 mmHg>P_{B}>3 mmHg, and/or
   25 mmHg>P_{B1}>15 mmHg, and/or
   25 mmHg>P_{C}>15 mmHg, and/or
   15 mmHg>P_{D}>5 mmHg, and/or
   15 mmHg>P_{E}>5 mmHg, and/or
   25 mmHg>P_{F}>15 mmHg, and/r
   15 mmHg>P_{G}>10 mmHg, and/or
   10 mmHg>P_{H}>2 mmHg, and/or
   10 mmHg>P_{I}>2 mmHg, and preferably
   25 mmHg>P_{D′}>15 mmHg, and/or
   15 mmHg>P_{E′}>5 mmHg, and/or
   25 mmHg>P_{F′}>15 mmHg, and/or
   25 mmHg>P_{G′}>15 mmHg;
the legging, according to the first embodiment, also has one or more of the following optional and preferred features:
   10 mmHg>P_{B}>3 mmHg, and
   25 mmHg>P_{B1}>15 mmHg, and
   25 mmHg>P_{C}>15 mmHg, and
   15 mmHg>P_{D}>5 mmHg, and
   15 mmHg>P_{E}>5 mmHg, and
   25 mmHg>P_{F}>15 mmHg, and
   15 mmHg>P_{G}>10 mmHg, and
   10 mmHg>P_{H}>2 mmHg, and
   10 mmHg>P_{I}>2 mmHg, and preferably
   25 mmHg>P_{D′}>15 mmHg, and
   15 mmHg>P_{E′}>5 mmHg, and
   25 mmHg>P_{F′}>15 mmHg. and
   25 mmHg>P_{G′}>15 mmHg;
the legging, according to a second embodiment, has one or more of the following optional and preferred features:
   10 mmHg>P_{B}>3 mmHg, and/or
   30 mmHg>P_{B1}>20 mmHg, and/or
   30 mmHg>P_{C}>20 mmHg, and/or
   25 mmHg>P_{D}>15 mmHg, and/or
   25 mmHg>P_{E}>15 mmHg, and/or
   20 mmHg>P_{F}>10 mmHg, and/or
   15 mmHg>P_{G}>10 mmHg, and/or
   10 mmHg>P_{H}>2 mmHg, and/or
   10 mmHg>P_{I}>2 mmHg. and preferably
   30 mmHg>P_{D′}>20 mmHg, and/or
   25 mmHg>P_{E′}>15 mmHg, and/or
   20 mmHg>P_{F′}>10 mmHg, and/or
   20 mmHg>P_{G′}>10 mmHg;
the legging, according to the second embodiment, also has one or more of the following optional and preferred features:
   10 mmHg>P_{B}>3 mmHg, and
   30 mmHg>P_{B1}>20 mmHg, and
   30 mmHg>P_{C}>20 mmHg, and
   25 mmHg>P_{D}>15 mmHg, and
   25 mmHg>P_{E}>15 mmHg, and
   20 mmHg>P_{F}>10 mmHg, and
   15 mmHg>P_{G}>10 mmHg, and
   10 mmHg>P_{H}>2 mmHg, and
   10 mmHg>P_{I}>2 mmHg. and preferably
   30 mmHg>P_{D′}>20 mmHg, and
   25 mmHg>P_{E′}>15 mmHg, and
   20 mmHg>P_{F′}>10 mmHg, and
   20 mmHg>P_{G′}>10 mmHg.

The invention also relates to a process for the treatment of OSAHS in which a product according to the invention is worn, preferably exclusively during the day, for more than 5 hours, preferably more than 8 hours, preferably more than 12 hours, or even more than 13 hours, and preferably continuously, i.e., without interruption.

The invention, according to another of its aspects, also relates to an elastic compression product for a wearer's lower limbs for use in the treatment of obstructive sleep apnoea-hypopnea syndrome, said product having a pressure profile defining textile pressures P_{B}, P_{B1}, P_{C}, P_{D}, P_{E}, P_{F}, P_{G}, P_{H}, P_{I}, P_{D′}, P_{E′}, P_{F′} and P_{G′} at altitudes B, B1, C, D, E, F, G, H, I defined in section 4.1.2 of standard NF_S_97_114, and at altitudes D′, E′, F′ and G′ located respectively at 92% of altitude D, at 110% of altitude E, at 126% of altitude E and at 92% of altitude G, respectively, the pressures being measured as the compression pressures of compression stockings according to French standard NF G 30-102 part B, the pressure profile being such that
35 mmHg>P_{C}>10 mmHg,
30 mmHg>P=_{F}>10 mmHg, and
P_{C}>(P_{B}+10 mmHg).

### BRIEF DESCRIPTION OF THE DRAWINGS

Other features and advantages of the invention will also appear upon reading the detailed description which follows, and upon examining the appended drawing wherein:

FIG. 1 schematically represents the morphological reference used to define the altitudes at which the textile pressures are measured;

FIG. 2 schematically illustrates a pressure profile according to a first example of a legging-type product according to the invention;

FIG. 3 schematically represents a pressure profile according to a second example of a legging-type product according to the invention;

FIG. 4 schematically illustrates a pressure profile according to a first example of a sock forming part of a sock/shorts-type product according to the invention;

FIG. 5 schematically represents a pressure profile according to a second example of a sock forming part of a sock/shorts-type product according to the invention;

FIG. 6 schematically illustrates a pressure profile according to a first example of a pair of shorts forming part of a sock/shorts-type product according to the invention; and

FIG. 7 schematically represents a pressure profile according to a second example of a pair of shorts forming part of a sock/shorts-type product according to the invention.

### DEFINITIONS

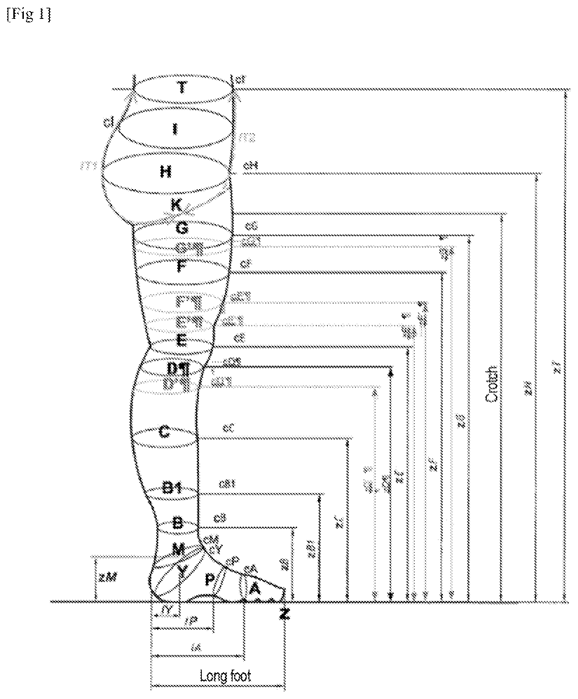

“Altitude” corresponds to a level in the vertical direction z when the compression product is worn by a wearer standing upright, as represented in FIG. 1.
As illustrated in FIG. 1, the altitudes of the lower limb are defined by the standard morphological reference, recalled in French standard NF_S_97_114. They have been represented, using the standard notations:
   B: ankle, at the point of its minimum circumference;
   B1: junction point between the Achilles tendon and the calf muscles;
   C: calf, at the point of its maximum circumference;
   D: just below the tibial tuberosity;
   E: in the centre of the patella and above the back of the knee (i.e., at the popliteal fossa);
   F: in the middle of the thigh;
   G: in the upper thigh;
   H: widest lateral trochanteric projections of the buttocks; and
   I: hips, at the level of the anterior superior iliac spines.
Furthermore, for the purposes of the invention, new altitudes have been defined:
   D′: located at 92% of altitude D;
   E′: located at 110% of altitude E;
   F′: located at 126% of altitude E; and
   G′: located at 92% of altitude G.
The “pressure profile” is defined by the change, along a compression product, of the textile pressure exerted by the product placed on a morphological template.
“Compression” is the effect produced by an elastic product worn on a person's lower limb, both at rest and during exertion, due to the restoring forces of the elastic fibres of this product.
The “textile pressure” is the pressure defined by French standard NF G 30-102 of October 1986, part B, called “compression pressure” and measured on a “compression stocking” in this standard. The procedure is described in section 8.1 of this part B of the standard. Reference is made to the application circumference C₁, i.e., the average of the application limit dimensions indicated by the manufacturer.
The coefficient of deformation A is defined by:

$$\frac{( {C}_{1} - {C}_{0} )}{{C}_{0}} \times 100 \qquad [ Math 1 ]$$

Δ is an application elongation percentage where C₀ is the circumference of the stocking flat.
In the context of the present invention, the values of A at different altitudes are given in the following tables. The first table concerns the kit and the second table concerns the legging.

TABLE 1

| | B | B1 | C | D′ | D | E′ | F′ | F | G′ | G | H | I |
|---|---|---|---|---|---|---|---|---|---|---|---|---|
| Δ | 29 | 71 | 92 | 59 | 54 | 66 | 63 | 63 | 69 | 73 | 78 | 82 |

TABLE 2

| | B | B1 | C | D′ | D | E | E′ | F′ | F | G′ | G | H | I |
|---|---|---|---|---|---|---|---|---|---|---|---|---|---|
| Δ | 29 | 71 | 92 | 59 | 54 | 60 | 66 | 63 | 63 | 69 | 73 | 78 | 82 |

The standard uses a “Hohenstein-type” leg model made of cone trunks, which is a standard in the field of support stockings.
When a product does not extend to an altitude, it is considered to exert zero textile pressure at that altitude. For example, a product according to the invention does not necessarily extend to altitude I.
The mmHg is commonly used as a textile pressure measuring unit in the field of medical compression. It can be easily converted to hPa:
   2 mmHg: roughly 2.66 hPa;
   4 mmHg: roughly 5.33 hPa;
   5 mmHg: roughly 6.66 hPa;
   8 mmHg: roughly 10.66 hPa;
   10 mmHg: roughly 13.3 hPa;
   12 mmHg: 16 hPa;
   15 mmHg: 20 hPa;
   20 mmHg: roughly 26.66 hPa;
   23 mmHg: roughly 30.66 hPa;
   25 mmHg: roughly 33.33 hPa;
   28 mmHg: roughly 37.33 hPa;
   30 mmHg: or 40 hPa.

Unless otherwise specified, a “pressure” is a textile pressure.

### DETAILED DESCRIPTION

The textile pressure profile of the compression product is decisive for its efficacy in treating OSAHS. The study of the pressure profile alone is however insufficient and wear tests are essential to verify that the compression product is comfortable. Failure to do so will result in poor compliance and reduced efficacy.

Numerous pressure profiles were thus tested and led to the selection of the products according to the invention.

The preferred pressure profile features of these products are listed below:
the pressure profile has a local maximum pressure, preferably an absolute maximum pressure, between altitudes B1 and D′, preferably at altitude C;
the pressure profile has a local maximum pressure between altitudes F′ and G′, preferably at altitude F;
the pressure profile has a minimum pressure (absolute minimum) between altitudes D and E′, preferably at altitude E;
the pressure profile has a local minimum pressure between altitudes D and E′, preferably at altitude E and/or at altitude E′;
the pressure profile has a local minimum pressure between altitudes H and 1;
the pressure profile has a local minimum pressure between altitudes B and B1, preferably at altitude B;
the pressure profile increases from altitude B to altitude B3;
the pressure profile increases from altitude E′ to altitude F′;
the pressure profile decreases from altitude D′ to altitude D;
the pressure profile decreases from altitude G′ to altitude G;
the pressure profile has a plateau from altitude B1 to altitude D′;
the pressure profile has a plateau from altitude F′ to altitude G′;
the pressure profile has a plateau from altitude H to altitude I;
the pressure profile has two pressure peaks, at the calf and at the thigh;
P_{B}<15 mmHg, preferably P_{B}<12 mmHg, preferably P_{B}<10 mmHg, preferably P_{B}<8 mmHg, and/or P_{B}>2 mmHg, preferably P_{B}>4 mmHg;
P_{B1}<30 mmHg, preferably P_{B1}<28 mmHg and/or P_{B1}>15 mmHg, preferably P_{B1}>20 mmHg;
P_{C}<30 mmHg, preferably P_{C}<28 mmHg and/or P_{C}>15 mmHg, preferably P_{C}>20 mmHg;
P_{D′}<30 mmHg, preferably P_{D′}<28 mmHg and/or P_{D′}>15 mmHg, preferably P_{D′}>20 mmHg;
P_{D}<15 mmHg, preferably P_{D}<12 mmHg, and/or preferably P_{D}>5 mmHg, preferably P_{D}>8 mmHg;
P_{E}>1 mmHg and/or P_{E}<25 mmHg, preferably P_{E}<20 mmHg, preferably P_{E}<15 mmHg, preferably P_{E}<10 mmHg, preferably P_{E}<5 mmHg;
preferably, P_{E}=0;
in particular for the kit of a product according to the invention including a sock and a pair of shorts, preferably P_{E′}>1 mmHg, preferably P_{E′}>5 mmHg and/or P_{E′}<25 mmHg, preferably P_{E′}<20 mmHg, preferably P_{E′}<15 mmHg;
P_{F′}<30 mmHg, preferably P_{F′}<25 mmHg, preferably P_{F′}<20 mmHg, and/or P_{F′}>10 mmHg, preferably P_{F′}>13 mmHg, or even P_{F′}>15 mmHg;
P_{F}<30 mmHg, preferably P_{F}<25 mmHg, preferably P_{F}<20 mmHg, and/or P_{F}>10 mmHg, preferably P_{F}>13 mmHg, or even P_{F}>15 mmHg;
P_{G′}<30 mmHg, preferably P_{G′}<25 mmHg, preferably P_{G′}<20 mmHg, and/or P_{G′}>10 mmHg, preferably P_{G′}>13 mmHg, or even P_{G′}>15 mmHg;
P_{G}<25 mmHg, preferably P_{G}<20 mmHg, preferably P_{G}<15 mmHg, and/or P_{G}>5 mmHg, preferably P_{G}>8 mmHg, preferably P_{G}>10 mmHg;
P_{H}<10 mmHg, preferably P_{H}<8 mmHg, and/or P_{H}>2 mmHg, preferably P_{H}>4 mmHg;
P_{I}<10 mmHg, preferably P_{I}<8 mmHg, and/or P_{I}>2 mmHg, preferably P_{I}>4 mmHg;
P_{C}>P_{F}, preferably P_{C}>(P_{F}+5 mmHg), preferably P_{C}≥(P_{F}+8 mmHg);
P_{E}<P_{C}, preferably P_{E}<P_{C}−5 mmHg, preferably P_{E}<P_{C}−10 mmHg, or even P_{E}<P_{C}−15 mmHg, or even P_{E}<P_{C}−20 mmHg, or even P_{E}<P_{C}−25 mmHg, provided that P_{E}≥0 mmHg;
   |P_{D}−P_{E}|<5 mmHg, preferably |P_{D}−P_{E}|<2 mmHg;
   |P_{C}−P_{B1}|<5 mmHg, preferably |P_{C}−P_{B1}|<2 mmHg;
P_{E}<P_{F}, preferably P_{E}<P_{F}−5 mmHg, preferably P_{E}<P_{F}−10 mmHg, or even P_{E}<P_{F}−15 mmHg, or even P_{E}<P_{F}−20 mmHg, or even P_{E}<P_{F}−25 mmHg, provided that P_{E}≥0 mmHg;
P_{B}<15 mmHg and (15 mmHg<P_{C}<30 mmHg) and P_{E}<15 mmHg and (10 mmHg<P_{F}<25 mmHg) and P_{H}<10 mmHg;
P_{B}<10 mmHg and (20 mmHg<P_{C}<30 mmHg) and P_{E}<10 mmHg and (12 mmHg<P_{F}<23 mmHg) and P_{H}<7 mmHg;
P_{B}<7 mmHg and (23 mmHg<P_{C}<27 mmHg) and P_{E}<5 mmHg and (12 mmHg<P_{F}<23 mmHg) and P_{H}<7 mmHg.

In general, the pressure profile of these products is irregular, with low pressure in the ankle, knee and pelvis, and moderate pressure in the calf and thigh.

In an embodiment, the product is open-footed, i.e., it does not cover the wearer's feet.

Preferably, the product is closed-footed.

A low pressure at the ankle makes it easier to put the product on, without affecting the product's efficacy. Preferably, 2 mmHg<P_{B}<15 mmHg.

A low pressure P_{H} at the pelvis, preferentially comprised between 2 mmHg and 8 mmHg, improves the comfort of wearing the product, without affecting its efficacy.

In an embodiment, the product is a legging.

In an embodiment, which is not preferred, in particular when the product is a legging, P_{E′}>P_{F}. Particularly for a legging, P_{E′} and/or P_{E} and/or P_{D} can be greater than 10 mmHg, greater than 15 mmHg, and preferably less than 25 mmHg.

For a legging, preferably P_{E} can be greater than 5 mmHg or even greater than 8 mmHg.

However, unexpectedly, the inventors discovered in particular that a low pressure at the knee does not significantly impair efficiency, while providing remarkable comfort. In particular, wear tests have shown, surprisingly, that no oedema forms at the knee.

In a preferred embodiment, the product is a kit composed of a pair of socks and a pair of shorts. The pressure at the knee P_{E} is advantageously zero, which ensures maximum comfort.

Preferably, the socks extend at least from altitude B to altitude D and/or the shorts extend at least from altitude E′, to altitude G, preferably to altitude H, preferably to altitude I.

The pressure profile for a sock is preferably such that the pressure decreases from altitude D′ to altitude D and such that P_{D}>8 mmHg and P_{D′}>23 mmHg.

The pressure profile for the shorts is preferably such that the pressure is maximum between the altitudes F′ and G′ and preferably such that P_{G}<P_{G′}.

According to a preferred embodiment, the compression product includes:
a sock extending from altitude B to altitude D, said sock having a pressure profile increasing from altitude B to altitude B1 and decreasing from altitude D′ to altitude D, the pressure profile of the sock having a maximum between altitudes B1 and D′, preferably a plateau between altitudes B1 and D′, the pressure P_{B} preferably being such that P_{B}≤10 mmHg, or even P_{B}≤5 mmHg, the pressure P_{C} preferably being between 20 mmHg and 35 mmHg, the pressure P_{D} preferably being such that P_{D}<15 mmHg, or even P_{D}≤10 mmHg, preferably (P_{D}+5 mmHg)<P_{C}, and
a pair of shorts extending from altitude E′ to altitude I, said shorts having a pressure profile increasing from altitude E′ to altitude F′ and decreasing from altitude G′ to altitude H, the pressure profile of the shorts having a maximum between altitudes F and G′, preferably a plateau between altitudes F′ and G′, and a minimum between altitudes H and I, preferably a plateau between altitudes H and 1, the pressure P_{E′} being preferentially such that P_{E′}<15 mmHg, or even P_{E′}≤10 mmHg, the pressure P_{F} being preferably comprised between 10 mmHg and 25 mmHg, again preferably between 10 mmHg and 20 mmHg, the pressure P_{H} being such that P_{H}<10 mmHg, or even P_{H}≤5 mmHg. (P_{E′}+5 mmHg)<P_{F}, preferably (P_{E′}+10 mmHg)≤P_{F}.

The manufacture of a product according to the invention does not pose any particular difficulty. In particular, the techniques usually used for the manufacture of elastic venous compression orthoses can be used.

The structure of a product according to the invention preferably comprises a network of stitches made by means of a knitting yarn, a weft yarn being interlaced between said stitches. The dimensioning and nature of the knitting and weft yarns as well as the stitch structure are chosen in such a way as to exert in the circumferential direction, after the product has been placed on the lower limb, an elastic restoring force capable of producing the desired textile pressure.

The different parts of the product according to the invention are preferably knitted continuously on a circular loom, with a single knitting yarn along the entire length of the product and with different weft yarns according to the zones considered and/or by varying the tightness of the knitted stitch.

The product can alternatively be made on a straight loom or a “cut-and-sew” loom.

In a preferred embodiment, all parts of the product are compressive.

Preferably, the went yarn is elastic, preferably is a double-covered elastane yarn.

In the case where the product is a legging, preferably:
the panty is made with two alternating knitting yarns C1 and C2, preferably C1 being in Lycra 44DT DG 1/44/34 and C2 being in Lycra 44DT DG 1/44/12,
the panty-thigh transition zone is made from a 1×3 weft and an alternating 1×3 bare Lycra, the bare Lycra advantageously producing a gripping zone and thus ensuring that the legging doesn't fall; the knitting being done preferentially on a covered 1×3 weft and on the bare Lycra with a knitted needle and three floating needles; the alternation between covered weft and bare Lycra advantageously modulates the gripping; the greater the bare Lycra surface in contact with the skin, the greater the gripping effect; the alternation as well as the number of knitted/floated needles being possible with many configurations.
the thigh/knee transition zone is made from a 1×3 weft and an alternating 1×3 bare Lycra,
the knee is made from a 2-weft comfort zone, the comfort zone being a zone where the stitch height is increased to provide great extensibility. In this case, the comfort zone has been extended to the back of the knee.
the knee/calf, calf and calf/ankle transition zones are made from two wefts,
the ankle is made from one weft,
the belt is made with a “boxer”-type elastic: but other alternatives are also possible, the reinforcement of the belt seam being preferentially made with a Text 1/44/34 thread.
the leg is made of two alternating weft threads J1 and J2, preferably J1 being in Lycra 570DT DG 1/22/7 and J2 being in Lycra 570DT Nu,
the knitting of the leg is in Lycra 17DT SG 1/85/92, and
the heel is made of Lycra 44DT DG 1/44/34 (800).

In the case where the product is a thigh/sock kit, preferably:
the panty is made of two alternating knitting yarns C1 and C2, preferably C1 being in Lycra 44DT DG 1/44/34 and C2 being in Lycra 44DT DG 1/44/12,
the panty/thigh transition zone is made from a 1×1 weft and a 1×3 bare Lycra, the bare Lycra advantageously producing a gripping zone and thus ensuring that the product doesn't fall at the level of the thigh; the knitting being done preferentially on a covered 1×1 wet and on 1×3 bare Lycra so as to ensure a maximum gripping effect, with a knitted needle and three floating needles; the greater the bare Lycra surface in contact with the skin, the greater the gripping effect; the alternation and the number of knitted/floated needles being possible with many configurations,
the thigh/knee transition zone is made from a 1×1 weft and a 1×3 bare Lycra,
the BC (side edge)/calf transition zones, the calf and the calf/ankle transition zone are made from two wefts,
the ankle and the foot are made from one weft,
the ends are made with “boxer”-type elastics; but other alternatives are also possible, the reinforcement of the belt seam being preferentially made with Text 1/44/34 yarn,
the leg for the shorts is made of two alternating weft yarns J1 and J2, preferably J1 being in Lycra 475DT DG 1/22/7 and J2 being in Lycra 475DT Nu,
the leg knit for the shorts is in Lycra 17DT SG 1/85/92,
the weft of the sock is made of Lycra 570DT DG 1/22/7,
the leg knit for the sock is in Lycra 17DT SG 1/85/92,
the heel is made of Lycra 44DT DG 1/78/23 Tex, and
the reinforcement of the seam of the side edge is in Tex 1/44/34.
Alternatively, other polyamide (PA) and/or polyester (P_{E}) yarns can be used for the manufacture of the shorts or the sock.
Each end of the shorts/thigh kit can be made according to different modes of construction to achieve the desired pressure, comfort and support objective. For example, wide “boxer”-type elastics, weft yarns of different cross-sections, and/or constructions combining more or fewer weft yarns per row on the outside or inside of the ends can be used.

### Examples

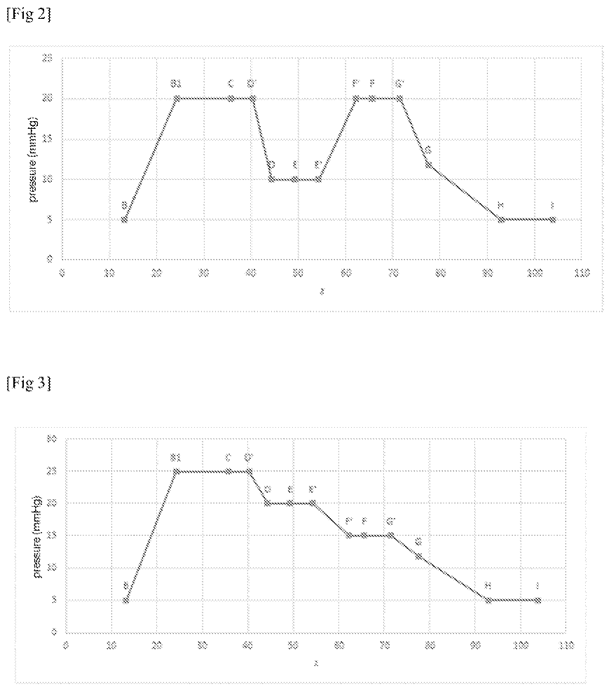

The graphs in FIGS. 2 to 7 represent the textile pressure in mmHg of different compression products according to the invention, as a function of altitude z. All the pressure profiles illustrated in these figures are segmented, but the pressure profile of a product according to the invention may also have another curve shape.

FIG. 2 illustrates an example of the pressure profile of a legging having two equal maxima at the calf and at the thigh, respectively, and more precisely a first plateau between altitudes B1 and D′ and a second plateau between altitudes F′ and G′, at a pressure of 20 mmHg. A minimum pressure is reached at 5 mmHg, especially at altitude B and at the pelvis with a plateau at 5 mmHg between altitudes H and L. A local minimum pressure in the form of a plateau is reached at the knee, between altitudes D and E′.

FIG. 3 shows an example of a legging pressure profile. This profile is not preferred. The pressure is at 5 mmHg at the ankle at altitude B, increases continuously until reaching a maximum at 25 mmHg between altitudes B1 and D′, then decreases continuously to return to 5 mmHg at altitude I, passing through two plateaus, a first plateau at 20 mmHg at the knee between altitudes D and E′ and a second plateau at 15 mmHg at the thigh between altitudes F′ and G′. At the pelvis, the pressure profile shows a minimum in the form of a plateau between altitudes H and I.

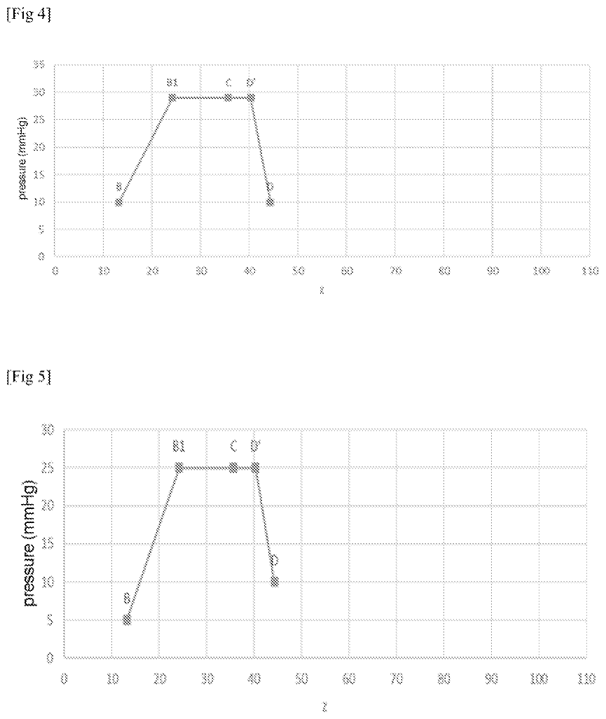

FIGS. 4 and 5 illustrate examples of sock pressure profiles where a maximum pressure in the form of a plateau, at 28 mmHg for FIG. 4 and at 25 mmHg for FIG. 5, is reached at the calf between altitudes B1 and D′, and the pressure at the knee at altitude D is at 10 mmHg. The functions of the two graphs increase continuously from B to B1 and decrease continuously from D′ to D. The two functions differ in the pressure value at altitude B and in the slopes of the straight lines connecting B to B1 and D′ to D, respectively. In the graph of FIG. 4, the pressure at altitude B is at 10 mmHg whereas it is at 5 mmHg in the graph of FIG. 5. The function of the latter has a steeper growth slope than that of the graph of FIG. 4.

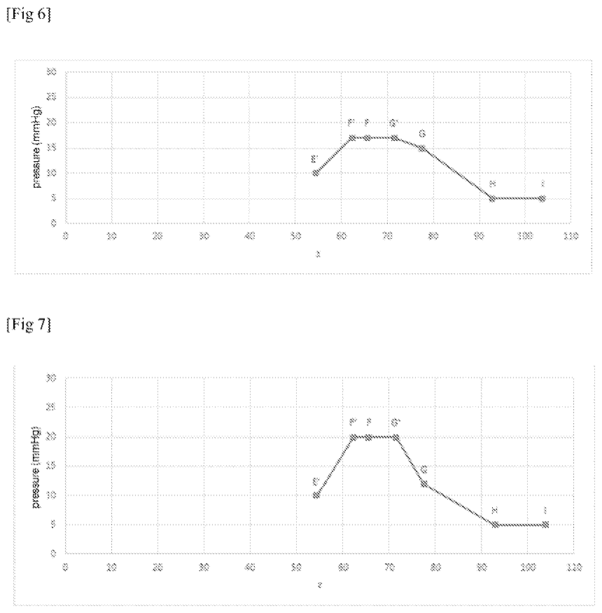

FIGS. 6 and 7 represent examples of shorts pressure profiles where a minimum pressure in the form of a plateau at 5 mmHg is reached at the pelvis between altitudes H and I, the pressure at the knee at altitude E′ being equal to 10 mmHg. The functions of the two graphs increase continuously from E′ to F′ to reach a maximum in the form of a plateau from F′ to G′, then decrease continuously from G′ to G and from G to H. The two functions differ by the value of the maximum and by the slopes of increase and decrease. In the graph of FIG. 6, the maximum is reached at 15 mmHg, whereas in the graph of FIG. 7, the maximum is at 20 mmHg. Furthermore, the graphs possess two slopes of decrease, the first being that of the line connecting G′ to G and the second being that of the line connecting G to H. The slope of the line (GG′) is steeper in the graph of FIG. 6 than the slope of the line (GG′) in the graph of FIG. 7, whereas the slope of the line (GH) is steeper in the graph of FIG. 7 than the slope of the line (GH) in the graph of FIG. 6.

To form a kit according to the invention, the sock having the profile illustrated in FIG. 4 can be combined in particular with either the shorts having the profile illustrated in FIG. 6 or the shorts having the profile illustrated in FIG. 7. Similarly, the sock having the profile illustrated in FIG. 5 can be combined in particular with either the shorts having the profile illustrated in FIG. 6, or that of FIG. 7.

Of course, the invention is not limited to the embodiments described and represented, provided for illustrative purposes only.

1. An elastic compression product for a wearer's lower limbs, intended for the treatment of obstructive sleep apnoea/hypopnea syndrome, said product having a pressure profile defining textile pressures P_{B}, P_{B1}, P_{C}, P_{D}, P_{E}, P_{F}, P_{G}, P_{H}, P_{I}, P_{D′}, P_{E′}, P_{F′} and P_{G′} at altitudes B, B1, C, D, E, F, G, H, I, defined in section 4.1.2 of standard NF_S_97_114, and at altitudes D′, E′, F′ and G′ located respectively at 92% of altitude D, at 110% of altitude E, at 126% of altitude E and at 92% of altitude G, respectively, the pressures being measured as the compression pressures of compression stockings according to French standard NF G 30-102 part B, the pressure profile being such that
35 mmHg>P_{C}>10 mmHg,
30 mmHg>P_{F}>0 mmHg and
P_{C}>(P_{B}+10 mmHg).

2. An elastic compression product for a wearer's lower limbs for use in the treatment of obstructive sleep apnoea/hypopnea syndrome, said product having a pressure profile defining textile pressures P_{B}, P_{B1}, P_{C}, P_{D}, P_{E}, P_{F}, P_{G}, P_{H}, P_{I}, P_{D′}, P_{E′}, P_{F′} and P_{G′} at altitudes B, B1, C, D, E, F, G, H, I defined in section 4.1.2 of standard NF_S_97_114, and at altitudes D′, E′, F′ and G′ located respectively at 92% of altitude D, at 110% of altitude E, at 126% of altitude E and at 92% of altitude G, respectively, the pressures being measured as the compression pressures of compression stockings according to French standard NF G 30-102 part B, the pressure profile being such that
35 mmHg>P_{C}>10 mmHg,
30 mmHg>P_{F}>10 mmHg, and
P_{C}>(P_{B}+10 mmHg).

3. The product according to claim 1, the pressure profile having
a local minimum pressure at altitude B, and/or
a local maximum pressure between altitudes B1 and D′, and/or
a local maximum pressure between altitudes F′ and G′, and/or
a local minimum pressure between altitudes D and E′, and/or
a local minimum pressure between altitudes H and I.

4. The product according to claim 1, wherein
P_{C}>(P_{F}+8 mmHg).

5. The product according to claim 1, wherein
3 mmHg<P_{B}<15 mmHg, and
P_{E}<13 mmHg, and
P_{H}<10 mmHg.

6. The product according to claim 1, wherein
P_{C}>20 mmHg, and
30 mmHg>P_{F}>10 mmHg.

7. The product according to claim 1, wherein
P_{B}>2 mmHg, and/or
15 mmHg>P_{D}>5 mmHg, and/or
13 mmHg>P_{F}>5 mmHg, and/or
15 mmHg>P_{E′}>5 mmHg, and/or
10 mmHg>P_{I}>2 mmHg.

8. The product according to claim 1, wherein
P_{B1}>15 mmHg, and/or
P_{D′}>15 mmHg, and/or
25 mmHg>P_{F′}>10 mmHg, and/or
25 mmHg>P_{G′}>10 mmHg.

9. The product according to claim 8, wherein
28 mmHg>P_{B1}>20 mmHg, and/or
28 mmHg>P_{D′}>20 mmHg, and/or
23 mmHg>P_{F′}>13 mmHg, and/or
23 mmHg>P_{G′}>13 mmHg.

10. The product according to claim 1, not extending beyond altitude I.

11. The product according to claim 1, in the form of a kit including a pair of socks and a pair of shorts.

12. The product according to claim 11, wherein
15 mmHg>P_{B}>3 mmHg, and/or
35 mmHg>P_{B1}>20 mmHg, and/or
35 mmHg>P_{C}>20 mmHg, and/or
15 mmHg>P_{D}>5 mmHg, and/or
23 mmHg>P_{F}>15 mmHg, and/or
20 mmHg>P_{G}>10 mmHg, and/or
10 mmHg>P_{H}>2 mmHg, and/or
10 mmHg>P_{I}>2 mmHg.

13. The product according to claim 12, wherein
35 mmHg>P_{D′}>25 mmHg, and/or
15 mmHg>P_{E′}>5 mmHg, and/or
23 mmHg>P_{F′}>15 mmHg, and/or
23 mmHg>P_{G′}>15 mmHg.

14. The product according to claim 1 wherein
33 mmHg>P_{C}>25 mmHg.

15. The product according to claim 1, wherein
30 mmHg>P_{B1}, and/or
30 mmHg>P_{D′}.

16. The product according to claim 1, wherein
10 mmHg>P_{B}.

17. A process for treating obstructive sleep apnoea/hypopnea syndrome wherein a product according to claim 1 is worn for more than 5 hours.
